(19) United States
(12) Patent Application Publication
(10) Pub. No.: US 2008/0038156 A1
(43) Pub. Date: Feb. 14, 2008

(54) ELECTRONIC CANDLE

(76) Inventor: Jamison Jaramillo, Novi, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/836,825

(22) Filed: Aug. 10, 2007

Related U.S. Application Data
(60) Provisional application No. 60/822,244, filed on Aug. 13, 2006.

(51) Int. Cl. A61L 9/03; A61L 9/00

(52) U.S. Cl. 422/124; 422/123; 422/125

(57) ABSTRACT

An electronic candle includes a source of light which provides a spatially and temporally varying pattern of illumination which simulates a candle flame, a volatile release station which operates to release a volatile material such as an aromatic material from a cartridge, and a forced convection system which disperses the volatile material. The candle further includes a controller which coordinates the operation of the release of the volatile material and the light source so that the pattern of light produced by the source is coordinated with an attribute of the volatile material being released.

### CROSS-REFERENCE TO RELATED APPLICATIONS

This application claims priority of U.S. Provisional Patent Application Ser. No. 60/822,244 filed Aug. 13, 2006, entitled “Cordless Portable Electronic Candle Providing Interactive Mood Enhancing Ambient Light and Scent Distribution.”

### FIELD OF THE INVENTION

This invention relates generally to electrical illumination devices. More specifically, the invention relates to electrical illumination devices which are simulative of a burning candle and which are further operative to dispense a scent or other volatile material.

### BACKGROUND OF THE INVENTION

Candles have long been used as a source of illumination. In addition, it has been known to incorporate volatile materials such as scents, odor neutralizing materials and the like into candles so that these volatile materials will be released as the candle is burned. The soft light emitted by a burning candle has a pleasant and tranquilizing effect, and this effect is further enhanced in those instances where the candle is scented. As a consequence, and despite the widespread availability of electrical illumination, candles are still extensively used for illumination, particularly in those instances where the illumination is intended to create a particular mood or effect.

The open flame and hot wax of a burning candle can present significant hazards to both persons and property. In addition, sooty carbon emissions from candles can constitute a health and environmental hazard as well as a nuisance. As a consequence, the prior art has made a number of attempts to provide illumination devices which simulate burning candles but which avoid hazards and nuisances associated with open flames. In some instances, such simulated candles are further operative to release scents and other volatile materials. For example, U.S. Patent Application Publications 2005/0254248 and 2004/0037069 both show electronic candles having simulated flames. U.S. Patent Application Publications 2005/0169666, 2005/0169812 and 2004/0257798 all show simulated candles which further operate to emit a scent.

While a number of electronic candles have been implemented in the prior art, all of such prior attempts have been found to be less than satisfactory. The prior art simulated candles have not been found to provide a realistic flame having a three-dimensional flickering structure. In addition, prior art simulated candles cannot operate so as to coordinate the pattern of the simulated flame with the emission of aromas or other such volatile materials.

As will be explained in detail hereinbelow, the present invention provides a simulated electronic candle which generates a three-dimensional pattern of illumination having spectral and spatial characteristics simulative of a flame. In addition, the candle of the present invention includes a scent distribution and dispersal system which operates in coordination with the simulated flame so that the characteristics of the flame in terms of spectral distribution, spatial distribution and temporal distribution of illumination is coordinated with the dispensing and dispersal of the volatile material. In this manner, a user may selectively control the characteristics of an ambient environment. These and other advantages of the invention will be apparent from the drawings, discussion and description which follow.

### BRIEF DESCRIPTION OF THE INVENTION

Disclosed herein is an electronic illumination device which simulates the operation of a candle. The illumination device includes a light source which is operable to deliver illumination having at least two different spectral patterns. The light source can further operate to deliver spatial and temporal patterns of illumination and in this manner can provide for a three-dimensional simulated flame. The light source, in some embodiments, includes light emitting diodes, which may be RGB diodes of the type which include a red emitter, a green emitter and a blue emitter. These emitters may be separately activated to control the spectral emission of the light source. In some instances, the light source will include at least six of the RGB diodes. The candle further includes a volatile material release station which functions to contain and deliver a volatile material such as an aromatic material, an odor neutralizing material, a pheromone, a therapeutic material or the like. The volatile release station is configured to receive a cartridge which contains the volatile material. The cartridge may contain a single material or may contain a plurality of different materials. The volatile release station further includes a heater which is in thermal contact with the cartridge. The heater includes a heating element and typically includes a thermal mass such as an aluminum body which retains and conducts heat.

In most instances, the candle will further include a forced convection system which includes at least one fan and which operates to disperse the volatile material released from the cartridge. The candle will also include a controller which coordinates the operation of the light source, the volatile release station, and the convection system. The controller may be microprocessor based, and may control the various elements in accord with a predetermined pattern. The components of the candle are enclosed in a housing which is shaped and configured to have an aesthetically pleasing configuration. The housing may include a user interface which permits a user to program and control the operation of the candle. The candle may be powered by electrical mains current or it may be a cordless device powered by a battery, which may comprise a lithium battery. In some instances, the battery may be a rechargeable battery, and the candle may include a charging station which may be an inductively coupled contactless charging station.

In some instances, the candle may operate so as to control the pattern of illumination in accord with a particular volatile material disposed in the cartridge. In this regard, the cartridge may include machine readable data thereupon which correlates with the volatile material in the cartridge. The candle will include a reader which is operable to read the machine readable data from the cartridge and provide a control signal which is communicated through the controller which in turn controls the operation of the illumination source so that a particular aroma may be coordinated with a particular pattern of illumination. In some instances, the cartridge may include a plurality of discrete segments, each containing a different volatile material, and this cartridge may include separate bodies of machine readable data for each volatile material.

### BRIEF DESCRIPTION OF THE DRAWINGS

FIG. 1 is a perspective view of an embodiment of candle in accord with the present invention;

FIG. 2 is a cross-sectional view of the candle of FIG. 1;

FIG. 3 is a top plan view of a cartridge which may be used in the candle of FIGS. 1-2;

FIG. 4 is a top plan view of the heater which may be used in the FIGS. 1-2 embodiment; and

FIG. 5 is a top plan view of a heat sink which may be used in the FIGS. 1-2 embodiment.

### DESCRIPTION OF THE INVENTION

The present invention comprises an electronic candle which simulates the natural warm three-dimensional flickering light of a candle flame. It is also capable of providing a multitude of mood stimulating colors in static, shifting and random spectrums, each keyed to a particular scent. The candle may be implemented in a variety of configurations, and for purposes of illustration, it will be described with one specific embodiment. It is to be understood that the principles of the present invention are not limited to any one particular embodiment, and may be implemented in a variety of configurations.

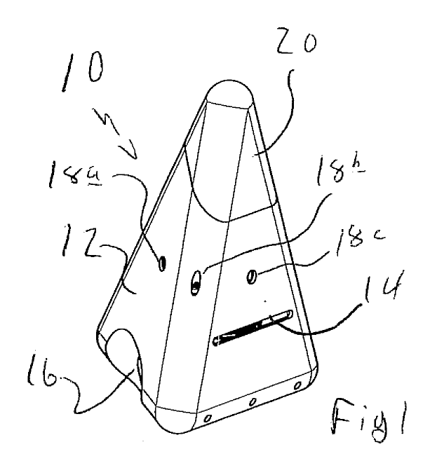

Referring now to FIG. 1, there is shown a perspective view of one embodiment of electronic candle 10. This candle 10 is configured in a general tetrahedral shape simulative of a pillar-type candle. The candle 10 includes a housing having a main body portion 12. Generally, this portion of the housing will be fabricated from an opaque material, and in particular instances the main portion of the housing 12 is fabricated from a polymeric material. As will be seen from FIG. 1, the main portion of the housing 12 includes a slot 14 on a front face thereof. This slot 14 is configured to receive a scent cartridge as will be explained in detail hereinbelow. The housing 12 includes an air inlet 16 proximate its base, and a series of air outlets 18a-18c. As will be explained in detail hereinbelow, the inlet and the outlets are part of a convection system which delivers scent or other volatile material from a cartridge which is placed in the slot 14.

The housing includes a top portion 20 fabricated from a transparent or translucent material. This portion is configured to contain a light source which is operated to simulate the candle flame. The top portion of the housing 20 is preferably fabricated from a polymeric material having good optical transparency. The outer surface of the housing 20 may be textured or otherwise configured to aid in the dispersal of light therefrom.

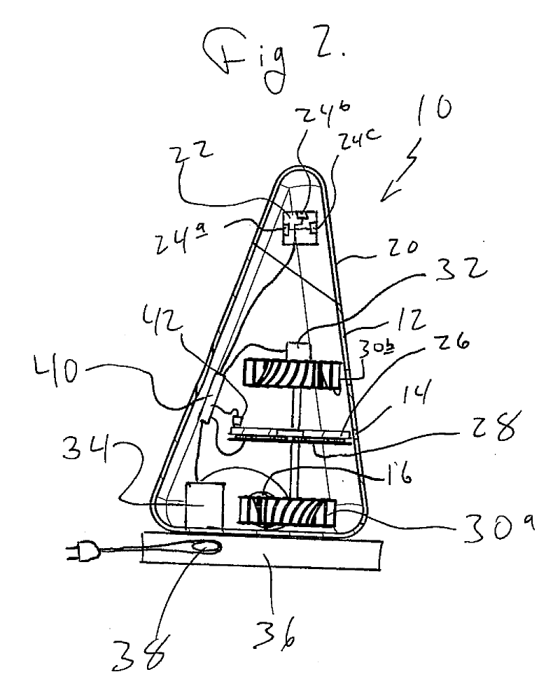

Referring now to FIG. 2, there is shown a cutaway view of an electronic candle generally similar to that shown in FIG. 1. The candle 10 of FIG. 2 includes a housing having a lower portion 12 fabricated from a generally opaque material such as a polymeric material as discussed above. The housing further includes an upper, transparent or translucent portion 20, which as discussed above is also generally fabricated from a polymeric material. As will be seen from FIG. 2, the candle 10 includes a light source 22 disposed in the upper portion 20 of the housing. This light source 22 is operative to provide illumination having at least two different visible patterns. For example, the illumination from the light source 22 may have at least two different spectral distributions; that is to say, the illumination may be of different colors. Also, the illumination may have different spatial patterns. For example, the illumination provided by the source 22 may have regions of different colors and/or different intensities. Furthermore, the light source 22 may provide a light which in whole or in part flickers. This combination of changing color, spatial distribution, intensity variation, and flicker pattern will simulate a natural flame. In addition, a light source having such capabilities will also be able to provide illumination patterns not achievable with a natural flame. By the appropriate combination of light patterns, various ambient environments can be created.

In one particular group of embodiments, the light source 22 includes light emitting diode (LED) light sources. As is known in the art, LED light sources are solid state semiconductor devices which are capable of providing high intensity, spectrally pure illumination. In addition, LED light sources are cool in operation, and very energy efficient. One particular type of LED light source having utility in the present invention is a device known as an RGB, full spectrum LED light source. Light sources of this type are comprised of three, and in some cases more, light emitting diodes, each of which emits a different wavelength of light. The combination of light from the three or more diodes produces a full spectrum, white appearing light. The individual diode portions comprising the RGB diode may be separately addressed so that the output of the light source may be varied. In this manner, the different wavelengths may be mixed and blended by controlling the intensity of the various emissions. In the embodiment of FIG. 2, three RGB LEDs 24a, 24b and 24c are included in the light source 22.

As previously discussed, the candle of the present invention is also capable of emitting and dispersing a volatile material such as a scent, pheromone, odor neutralizing agent or the like. In this regard, the candle 10 of FIG. 2 is configured to accept a cartridge 26, which may be inserted through the cartridge slot 14 in the lower housing 12. The cartridge 26 may include one or more volatile materials therein, and in typical embodiments, the volatile materials are dissolved or dispersed in a waxy carrier. Details of the cartridge will be discussed hereinbelow.

Also included in the candle 10 is a cartridge heater 28. This heater 28 is operative to warm the cartridge 26 and activate the release of the volatile material therefrom. The heater is typically an electrical heater, and may have a heat sink/reservoir associated therewith. This heat sink/reservoir may be comprised of a body of aluminum or other such metal. In one instance, the heater is regulated so as to heat the cartridge to a temperature which is less than the temperature at which the waxy carrier for the volatile material melts. This avoids the hazards of molten material.

The electronic candle 10 further includes a forced convection system which operates to disperse material which is volatilized from the cartridge 14. In the illustrated embodiment, the forced convection system is comprised of a pair of fan turbines 30a, 30b disposed so as to create a flow of air from the air intake 16, through the lower housing 12, and out the outlets 18. In the illustrated embodiment, both turbines 30a, 30b are turned on a common shaft by an associated electric motor 32; although, it is to be understood that other convection systems may be implemented. For example, a single fan or more than two fans may be used to disperse the volatile material.

The candle 10, in this embodiment, is powered by a battery 34, which may comprise a rechargeable battery such as a lithium ion battery, a nickel metal hydride battery, a nickel cadmium battery or the like. In other instances, the battery 34 may be a replaceable battery. In the illustrated embodiment, the battery 34 is a rechargeable battery, and as illustrated, the candle 10 includes a base station 36 having an inductive battery charger 38 built thereinto. In this manner, the base station 36 is operable to recharge the battery 34 from mains current. Other charger arrangements are also contemplated. Furthermore, in some instances, the battery may be dispensed with and the candle 10 configured to run directly from mains current.

The candle of FIG. 2 further includes a controller 40 disposed in the housing. This controller is in electrical communication with the light source 22, the fan motor 32, the heater 28, and the battery power source 34. The controller may be a microprocessor-based controller, and it operates to apportion power to the various elements of the candle. In this regard, the controller 40 includes stored programs for activating the light source 22 so as to provide a spatially and temporally varying light pattern simulative of a candle flame. The controller 40 is also operative to control the heating of the cartridge 14, and the activation of the motor 32 so as to disperse volatile material from the cartridge.

In the illustrated embodiment, the candle 10 further includes a reader device 42 disposed in proximity to the cartridge 14. In this embodiment, the cartridge 26 includes machine readable data encoded thereupon which correlates with a specific volatile material disposed in the cartridge. The reader 42 reads this machine readable data and communicates a signal to the controller 40 which in turn uses this data to control the operation of the light source 22. In this manner, the candle will provide a light pattern corresponding to the volatile material being dispersed. For example, a citrus aroma may cause the candle to emit a light pattern which has a large number of components in the yellow portion of the visible spectrum. A pine aroma can cause the simulated flame to have a greenish pattern, while lavender or other floral aromas can create a lilac tinged, simulated flame. In yet other instances, the further attributes of the simulated flame such as flicker rate, intensity, shape and the like may also be keyed into the nature of the volatile material. The machine readable signal may be an optical signal, in which instance the reader 42 may be a photodiode or the like. Machine readable data may also be written magnetically, and the reader may be a magnetic pickup. A signal may also be a mechanical signal as may be produced by a punched pattern or a shaped edge on the cartridge, and the reader may include mechanical probes which read this data.

In some instances, the cartridge may include a plurality of different volatile materials, and each material will have a discrete body of machine readable data associated therewith, and the heat source will be operable to selectively heat different portions of the cartridge so as to release a selected volatile material. This may be accomplished by using a segmented heater or by mechanically moving the disk via an electric motor, clockwork, or manual means. In some instances, the controller will be operable to activate the selective release of different volatile materials from a multi-material cartridge. In this manner, the candle may be programmed to produce a changing pattern of light and aroma so as to create a time variant ambient atmosphere. These patterns may be preprogrammed into the candle or may be user selected.

Referring now to FIG. 3, there is shown one embodiment of cartridge 28 which may be used in the candle of the present invention. As illustrated, the cartridge is a disk-shaped member which includes a plurality of discrete segments, for example segments 44 and 46. Each segment defines a well which retains a volatile material therein. The volatile material is, in one particular embodiment, retained in a body of hydrocarbon material such as wax, and in specific instances, soy-based wax has been found to be useful in the present invention. The volatile material, as noted above, may comprise aromatic compounds such as natural or synthetic essences. The volatile material may also comprise pheromones or other mood altering substances, or odor absorbing or neutralizing material such as diethylene glycol and the like. It is contemplated that blends of various volatile materials may be likewise utilized. The cartridge 28 of FIG. 3 is, in one embodiment, fabricated from a biodegradable material such as heavy paper stock. In other instances, the cartridge may be fabricated from metal foil, polymeric material, fibrous material or the like. While the cartridge 28 of FIG. 3 is shown as a circular member having a number of discrete wedge-shaped segments, it is to be understood that the cartridge may be otherwise configured. For example, cartridges may be of rectangular or triangular shape.

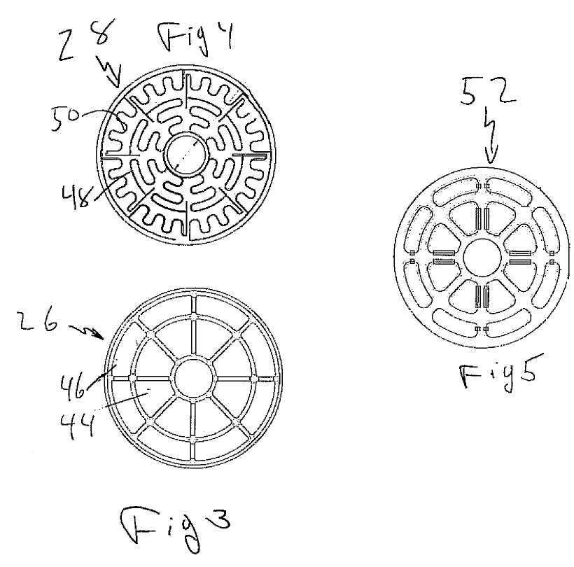

Referring now to FIG. 4, there is shown one embodiment of heater which may used in the present invention. This heater 28 is a multi-segment electrical resistance heater. In the illustrated embodiment, the heater 28 includes a plurality of discretely activatable segments, for example segments 48 and 50 which correspond to the segments of the cartridge 26 of FIG. 3. As will be understood, the activation of a specific segment of the heater 28 will cause a corresponding segment of the cartridge to be activated. By appropriate control of the heating circuit, it may be assured that the level of heat produced by the heater is less than that which will be required to melt the wax containing the volatile substances. In this manner, dangers associated with molten wax will be avoided.

Referring now to FIG. 5, there is shown a heat sink 52 which may be incorporated into the candle in combination with the heater 28 of FIG. 4. Although not essential to the present invention, it has been found that incorporation of a heat sink, particularly in a multi-segment cartridge embodiment, further enhances the operation of the scent dispersal system. The heat sink 52 is fabricated from a metal having good thermal conductivity such as aluminum. The heat sink 52 is configured in a pattern corresponding to the pattern of the heater 28 of FIG. 4 and the cartridge 26 of FIG. 3. In this manner, the heat sink operates to collect, focus and limit heat so that a single segment of the cartridge may be reliably and accurately activated, without causing release of volatile material from adjacent segments. It will be understood that one of skill in the art could readily implement various configurations of cartridge and heating assembly in view of the teaching presented herein, and all of such embodiments are within the scope of this invention.

In view of the foregoing, it will be understood that an electronic candle system of the present invention operates to produce a three-dimensional, time and color variant illumination pattern which may simulate an actual flame or provide a unique variation thereof. The candle further operates to release a volatile material, and the characteristics of the simulated flame may be controlled in accord with the nature of the released volatile material so as to establish an ambient atmosphere in a user's environment. The candle of the present invention may be programmed to produce a changing pattern of aroma and light and as such will have aesthetic as well as therapeutic value. In that regard, it is known in the art that particular aromatic materials do have health and mood altering qualities. Furthermore, volatile materials such as citronella, pyrethins, terpenes and the like can function as insect repellants and control agents. All of such materials may be used and dispensed by the candle system of this invention.

The foregoing drawings, discussion and description illustrate some specific embodiments of the invention, but are not meant to be limitations upon the practice thereof. Numerous modifications and variations thereof will be apparent to those of skill in the art, and it is the following claims, including all equivalents, which define the scope of this invention.

1. An electronic candle comprising:
a light source which includes at least three RGB LEDs disposed in a three-dimensional array, said light source being operable to provide a spatial and temporal pattern of illumination simulative of a candle flame;
a volatile release station, said station including a cartridge having at least one volatile material retained thereupon and a heater in thermal contact with the cartridge, said heater including an electrically activatable heating element;
a forced convection system operable to establish a flow of air for dispersing a volatile material released from said volatile release station;
a source of electrical power;
a controller in electrical communication with said source of power, said forced convection system, said volatile release station, and said light source, said controller being activatable to control the operation of at least one of said elements in accord with a predetermined program; and
a user interface in communication with said controller, said interface being operable to permit a user to selectably control the operation of said controller.

2. The candle of claim 1, wherein said power source is a battery.

3. The candle of claim 2, wherein said battery is a rechargeable lithium ion battery.

4. A simulated candle which provides for the coordinated emission of light and a volatile material, said candle comprising:
a light source which is operable to deliver illumination having at least two different spectral patterns;
a replaceable cartridge containing a volatile material, said cartridge including machine readable data corresponding to the volatile material in said cartridge;
a reader operable to read said machine readable data on said cartridge and provide a control signal in response thereto; and
a controller in communication with said reader and said light source, said controller being operable to select one of said at least two different spectral patterns in response to a specific control signal it receives from said reader so that the spectral pattern of light emitted by said light source corresponds to a particular volatile material in said cartridge.

5. The candle of claim 4, further including a heater for heating said cartridge so as to activate release of the volatile material therefrom.

6. The candle of claim 4, wherein said cartridge includes a plurality of sections, each section containing a different volatile material, said cartridge further including a plurality of bodies of machine readable data, each body of data corresponding to a different volatile material.

7. The candle of claim 6, further including a heater which is operable in combination with the cartridge to selectably heat one of said sections so as to activate the release of a volatile material from that section, and wherein said reader is operable to read the body of machine readable data associated with said section which is heated.

8. The candle of claim 7, wherein said heater is operable to sequentially heat different sections of said plurality of sections in said cartridge.

9. The candle of claim 4, wherein said cartridge is comprised of a body of cellulosic material.

10. The candle of claim 4, wherein in said cartridge, said volatile material is dispersed in a hydrocarbon wax.

11. The candle of claim 4, wherein said volatile material is selected from the group consisting of aromatic materials, odor masking materials, insecticides, therapeutic materials, pheromones, and combinations thereof.

12. The candle of claim 4, further including a fan operable to disperse said volatile material.

13. The candle of claim 4, wherein said light source includes at least one light emitting diode.

14. The candle of claim 13, wherein one of said at least one light emitting diode is an RGB light emitting diode which is comprised of a red emitter, a green emitter and a blue emitter.

15. The candle of claim 4, wherein said machine readable data is: electronically readable, magnetically readable, optically readable, or mechanically readable.

16. The candle of claim 4, wherein said light source is operable to deliver a flickering illumination.

17. The candle of claim 16, wherein said flickering illumination flickers in accord with at least one preselected pattern.

18. The candle of claim 15, wherein said preselected pattern is a three-dimensional pattern.

19. A simulated candle which provides for the coordinated emission of light and aroma, said candle comprising:
a light source which includes at least one light emitting diode, said source being operable to deliver illumination having at least two different spectral patterns;
a replaceable cartridge which includes a plurality of different aromatic materials supported thereupon, said cartridge being further configured to include a plurality of bodies of machine readable data, each body corresponding to a different volatile material;
a heater which is operable to selectably heat a particular aromatic material in the cartridge so as to selectably activate the release of said aromatic material;
a reader which is operable to read the body of data associated with the aromatic material which is heated, and provide a control signal in response thereto;
a controller which is in communication with the reader and the light source, said controller being operable to select one of said at least two different spectral patterns in response to a specific control signal it receives from the reader so that the spectral pattern of light emitted by the light source corresponds to the particular volatile material being heated in said cartridge; and
a fan which is operable to disperse the aromatic material being heated.
